(19) United States
(12) Patent Application Publication
(10) Pub. No.: US 2017/0276635 A1
(43) Pub. Date: Sep. 28, 2017

(54) FLEXIBLE AND TRANSPARENT GAS SENSOR BASED ON MOS2 AND METHOD FOR MANUFACTURING THE SAME

(71) Applicant: INDUSTRY-ACADEMIC COOPERATION FOUNDATION YONSEI UNIVERSITY, Seoul (KR)

(72) Inventors: Jongbaeg KIM, Goyang-si (KR); Dae-Hyun BAEK, Seoul (KR); Kyounghoon LEE, Seoul (KR); Soonjae PYO, Seoul (KR)

(21) Appl. No.: 15/188,412

(22) Filed: Jun. 21, 2016

(30) Foreign Application Priority Data
Mar. 28, 2016 (KR) 10-2016-0037230

(51) Int. Cl. G01N 27/407

(52) U.S. Cl. CPC G01N 27/4074; G01N 33/0047

(57) ABSTRACT

A gas sensor includes: an insulating substrate; a gas sensing portion immobilized on the substrate and comprising MoS₂ flakes containing metal porphyrin; and a Pair of electrodes formed at both ends of the MoS₂ flakes of the gas sensing portion so as to be spaced apart from each other. When MoS₂ flakes are functionalized using cobalt tetraphenylporphyrin as metal porphyrin, the sensitivities thereof to benzene and toluene are significantly increased.

### CROSS-REFERENCE TO RELATED APPLICATIONS

This application claims priority to Korean Patent Application No. 10-2016-0037230, filed Mar. 28, 2016, the entire content of which is incorporated herein by reference.

### BACKGROUND OF THE INVENTION

### 1. Field of the Invention

The present invention relates to a flexible and transparent gas sensor and a method for manufacturing the same, and more particularly, to a gas sensor comprising a molybdenum disulfide cluster functionalized with metal porphyrin so as to have a significantly increased sensitivity to specific volatile organic compounds (VOCs), and a method for manufacturing the same.

### 2. Description of Related Art

Volatile organic compounds (hereinafter also referred to as “VOCs”), including acetone, ethanol, acetaldehyde, toluene and benzene, exist in a gaseous state at room temperature to reduce the quality of indoor air and threaten human health. As one of conditions that are caused by VOCs, sick building syndrome is well known, which is caused by exposure to toxic compounds indoors.

Thus, the importance and need to detect VOCs by monitoring the indoor air quality are increasing.

It was reported that conventional VOC sensors may use tungsten oxide (Sens. Actuator B-Chem., vol. 108, pp. 97-101, 2005), tin oxide nanofibers (Sens. Actuator B-Chem., vol. 137, pp. 471-475, 2009), or titanium oxide-doped zinc oxide nanostructures (Sens. Actuator B vol. 140, pp. 73-78, 2009) as sensing materials to increase the sensor sensitivity.

However, the metal oxide-based sensors as described above can be operated at high temperatures (about 300° C.), and thus cannot be used at room temperature. For this reason, a separate device is required to maintain high temperatures at which the sensors can be operated.

In addition, because these VOC sensors can be operated only when they are maintained at high temperatures, transparent organic substrates cannot be used. For this reason, it is difficult to integrate the VOC sensors in wearable devices attracting attention as a next-generation technology, and it has been limited to use the VOC sensors in various manners.

Prior Art Documents related to the present invention include Korean Patent No. 10-1422625 (published on Jul. 24, 2014).

### SUMMARY OF THE INVENTION

It is, an object of the present invention to provide a gas sensor that can be miniaturized and, at the same time, comprises a flexible and transparent substrate, and a method capable of manufacturing the gas sensor in a simple, easy and cost-effective manner.

Another object of the present invention is to provide, a gas sensor having an increased selective sensitivity to specific volatile compounds, and a method for manufacturing the gas sensor.

To achieve the above objects, the present invention provides a gas sensor comprising: an insulating substrate; a gas sensing portion immobilized on the substrate and comprising MoS₂ flakes containing metal porphyrin; and a pair of electrodes formed at both ends of the MoS₂ flakes of the gas sensing portion so as to be spaced apart from each other.

In an, embodiment of the present invention, the gas sensing portion is formed of a MoS₂ cluster consisting of a plurality of the MoS₂ flakes.

In this embodiment, the pair of electrodes are formed at both ends of the gas sensing Portion so as to be spaced apart from each other at a distance at which they commonly come in contact with one or more of the MoS₂ flakes.

In a preferred embodiment, the pair of electrodes may be disposed so as to be spaced apart from each other at a distance equal to half or less of the average length of the MoS₂ flakes.

In addition, the substrate may be made of a flexible and transparent material such as PET.

In an embodiment of the present invention, the metal porphyrin may be cobalt tetraphenylporphyrin (Co-TPP).

In this case, the gas sensing portion has an increased sensitivity (ΔR/R₀) to benzene or toluene compared to a gas sensing portion formed of pristine MoS₂ flakes containing no cobalt tetraphenylporphyrin.

Herein, the sensitivity (ΔR/R₀) of the gas sensing portion may be at least 2 times higher to benzene and 30% higher to toluene.

The present invention also provides a method for manufacturing a gas sensor, comprising the steps of: (a) mixing a solution containing a plurality of MoS₂ flakes with a metal porphyrin-containing solution to prepare a mixture solution; (b) placing droplets of the mixture solution on an insulating substrate, and drying the Placed droplets, thereby forming a gas sensing portion; and (c) forming a pair of electrodes at both ends of the gas sensing so as to be spaced apart from each other at a distance at which they commonly come in contact with one or more of the MoS₂ flakes.

Herein, the pair of electrodes may be formed so as to be spaced apart from each other at a distance equal to half or less of the average length of the MoS₂ flakes. In this case, the pair of electrodes may be formed at both ends of the gas sensing portion in any direction.

Herein, the substrate may be made of a flexible and transparent material such as PET.

In an embodiment of the present invention, the metal porphyrin may be cobalt tetraphenylporphyrin (Co-TPP), and the gas sensing portion has an increased sensitivity to benzene or toluene compared to a gas sensing portion formed of a pristine MoS₂ cluster containing no cobalt tetraphenylporphyrin.

### BRIEF DESCRIPTION OF THE DRAWINGS

FIG. 1 shows a series of processes for manufacturing a gas sensor according to the present invention.

FIGS. 2A and 2B are SEM photographs of a pristine MoS₂ cluster and a MoS₂ cluster functionalized with cobalt tetraphenylporphyrin, respectively.

FIG. 3 shows the Raman spectrum of MoS₂ used in an example of the present invention.

FIG. 4 is a graph showing the light transmittance of a cobalt tetraphenylporphyrin-functionalized MoS₂ cluster disposed on a PET substrate as a function of wavelength.

FIG. 5A is a graph showing the sensitivity to benzene of a gas sensor comprising each of a pristine MoS₂ cluster and a MoS₂ cluster functionalized with cobalt tetraphenylporphyrin, and

FIG. 5B is a graph showing the sensitivity to benzene of each gas sensor as a function of the concentration of benzene.

FIG. 6 is a graph showing the sensitivity to toluene of a gas sensor comprising each of a pristine MoS₂ cluster and a MoS₂ cluster functionalized with cobalt tetraphenylporphyrin.

FIG. 7 shows the results of comparing sensitivity when bending deformation was applied to a gas sensor comprising a transparent and flexible substrate.

### DETAILED DESCRIPTION OF THE INVENTION

Hereinafter, preferred embodiments of the present invention will be described in detail with reference to the accompanying drawings.

In the following description, the detailed description of known configurations that are obvious to those skilled in the art will be omitted when it may obscure, the subject matter of the present invention. In the drawings, the thickness of lines or the size of constituent elements may be illustrated exaggeratingly for the clarity and convenience of description.

In addition, terms such as first, second, A, B, (a), (b), and the like may be used herein to describe components. Each of these terminologies is not used to define the essence, order or sequence of a corresponding component, but is used merely to distinguish the corresponding component from other component(s). It should be noted that if it is described in the specification that one component is “connected”, “coupled”, or “joined” to another component, a third component may be “connected”, “coupled”, and “joined” between the first and second components, although the first component may be directly connected, coupled or joined to the second component.

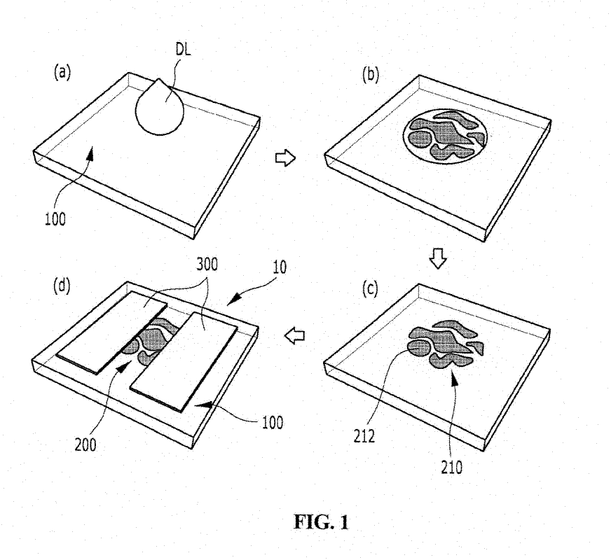

It is advantageous to understand a series of processes for manufacturing a gas sensor 10 (See FIG. 1) according to the present invention in order to understand the structure of the gas sensor 10. Thus, a method for manufacturing the gas sensor according to the present invention will now first be described with reference to FIG. 1.

As shown, in FIG. 1, the gas sensor 10 according to the present invention characterized in that it can be manufactured by a simple method comprising: placing an insulating substrate 100 droplets DL of a MoS₂ cluster 210 consisting of a plurality of functionalized MoS₂ flakes 212; drying the placed droplets; and forming a pair of electrodes at both ends of the dried MoS₂ cluster 210. Processes for manufacturing the gas sensor 10 according to the present invention will now be, described in detail.

First, a process of mixing a solution containing a plurality of MoS₂ flakes 212 with a metal porphyrin-containing solution to functionalize the MoS₂ flakes 212 is performed.

As used herein, the expression “functionalize the MoS₂ flakes 212” means increasing the sensitivity of the MoS₂ flakes 212 to specific VOCs compared to that of pristine MoS₂ flakes. The kind of VOCs to which sensitivity is to be increased is determined according to the kind of metal porphyrin. In an example of the present invention, cobalt tetraphenylporphyrin (Co-TPP) was used as metal porphyrin, and as a result, the sensitivity to benzene of the MoS₂ flakes 212 was dramatically increased. The results of this experiment will be described in detail later.

In an example of the present invention, a solution of the MoS₂ flakes 212 and a solution of cobalt tetraphenylporphyrin were all prepared using ethanol as a solvent. The concentration of the MoS₂ flakes 212 was 25 mg/f, and the concentration of cobalt tetraphenylporphyrin was 1.0 g/l. The two solutions were mixed at a ratio of 1:1, and the molecular weights of MoS₂ and Co-TPP were 160.07 and 671.65, respectively. After mixing, the mass ratio and molar ratio of MoS₂:Co-TPP were 1:40 and 1:9.5, respectively.

After the mixture solution of the MoS₂ flakes 212 and the metal porphyrin is prepared as described above, droplets DL of the mixture solution are placed on a flexible and transparent substrate 100, and then dried. In an example of the present invention, a flexible PET substrate 100 was used as the insulating substrate, and the droplets were dried at a temperature of 100° C.

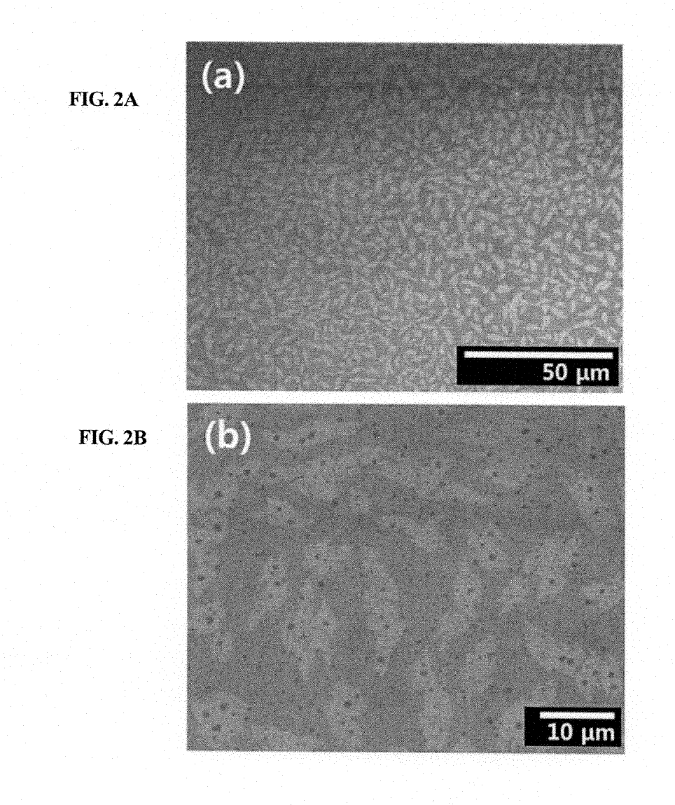

FIG. 2A is a scanning electron microscope (SEM) photograph of a pristine MoS₂ cluster placed and dried on a silicon substrate, and FIG. 2B is a SEM photograph of a cobalt tetraphenylporphyrin-functionalized MoS₂ cluster 210 placed and dried on a silicon substrate. In the photograph, the portions looking bright are MoS₂ flakes 212, and the spots looking gray darker than the portions are cobalt tetraphenylporphyrin (Co-TPP). As can be seen in the photograph, MoS₂ flakes 212 are very uniformly distributed in a state in which droplets DL of the mixture solution are dried. The dried MoS₂ cluster 210 placed on the substrate 100 functions as a gas sensing portion 200 for detecting VOCs.

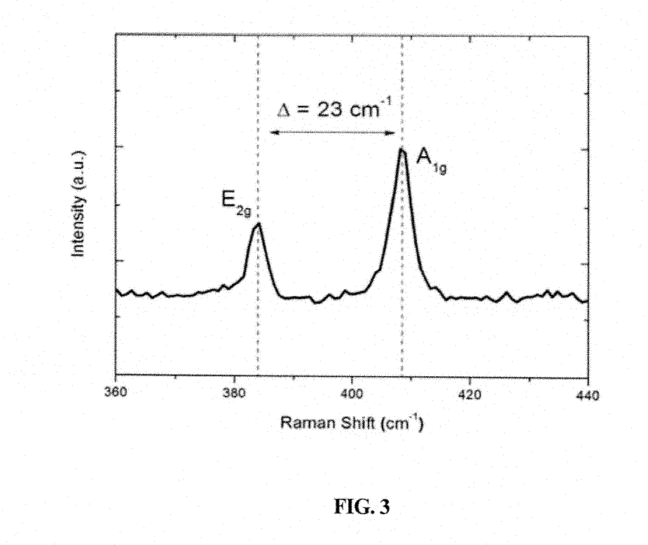

FIG. 3 is a graph, showing the Raman spectrum of the MoS₂ flakes 212 used. The spectrum was obtained by irradiating MoS₂ nanosheets with an argon ion laser (having a wavelength of 521 nm) with a spot diameter of 1 μm at an output of 0.5 mW.

It is possible to determine the number of MoS₂ layers by a Raman shift (Δ) indicating the distance between in-plane E_{2g} mode and out-of-plane A_{lg} mode. It is presumed that the MoS₂ flakes 212 used in the example of the present invention consist of a three-ply layer.

After the dried gas sensing portion 200 is formed on the substrate 100, a pair of electrodes is formed at both ends of the gas sensing portion 200 so as to be spaced apart from each other at a distance at which they commonly come in contact with one or more MoS₂ flakes 212. That is, a pair of electrodes spaced apart from each other is formed to share one or more MoS₂ flakes 212 so that a current can flow through the MoS₂ flakes 212. Herein, the electrodes may be made of a metal, for example, a chromium/gold (Cr/Au) alloy.

As shown in FIG. 2, the MoS₂ flakes 212 used have an average length of 10 μm. If a pair of electrodes are formed so m as to be spaced apart from each other at a distance of 5 μm (corresponding to half of the average length) or less, the electrodes will commonly come in contact with one or more MoS₂ flakes 212. This suggests that, if the average length of the MoS₂ flakes 212 is known, it is possible to manufacture the gas sensor 10 by forming a pair of electrodes so as to spaced apart from each other at a distance corresponding to half or less of the average length without having to precisely align the electrodes (in other words, by forming the electrodes at both ends of the gas sensing portion in any direction).

Thus, in the method for manufacturing the gas sensor according to the present invention, the gas sensor is manufactured by mixing the solutions to functionalize the MoS₂ flakes 212, dropping the droplets DL onto the substrate 100, drying the dropped droplets, and then forming a pair of electrodes so as to be spaced apart from each other at distance corresponding to half or less of the average length of the MoS₂ flakes 212. Thus, the method of the present invention is very suitable to produce a large amount of the gas sensor 10 in a cost-effective manner.

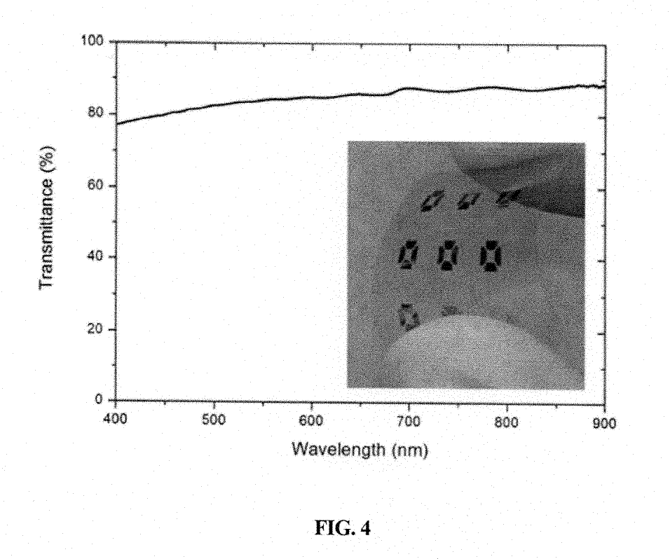

FIG. 1(d) schematically shows the structure of the manufactured gas sensor 10. The appearance of the actually manufactured gas sensor is shown in the insert of FIG. 4.

With reference to FIG. 1(d) and a series of manufacturing processes as described above, the configuration of the gas sensor 10 according to the present invention will now be described.

The above-described method for manufacturing the gas sensor 10 is a method designed so as to be suitable for mass production in commercial terms. Namely, it will be advantageous to form the gas sensing portion 200 using the MoS₂ cluster 210 in view of mass production, although the effect of increasing sensitivity can also be obtained for one MoS₂ flake 212 functionalized with metal porphyrin. In such terms, the configuration of the gas sensor 10 according to the present invention, which comprises the gas sensing portion 200 formed of the functionalized MoS₂ cluster 210, will be described hereinafter, it should be noted that the technical characteristic of the present invention is that specific VOCs are sensitively detected using the MoS₂ flakes 212 functionalized with metal porphyrin.

The gas sensor 10 according to the present invention comprises: a transparent and flexible substrate 100; and a gas sensing portion 200 immobilized on the substrate 100; and a pair of electrodes formed at both ends of the gas sensing portion 200.

In an example of the present invention, a PET substrate 100 was used as the transparent and flexible substrate 100. FIG. 4 is a graph showing the light transmittance of the manufactured gas sensor 10 as a function of wavelength. As can be, seen in FIG. 4, the gas sensor 10 showed a high light transmittance of about 80-90%. In addition, as shown in the insert of FIG. 4, the gas sensor 10 has a small size and also has a mechanical strength allowing it to be suitably bent. Thus, it can be seen that the gas sensor 10 has properties very suitable for application to wearable devices that are highly likely to be developed in the future.

The gas sensing portion 200 is formed of the MoS₂ cluster 210 consisting of a plurality of MoS₂ flakes 212 containing metal porphyrin. As described above, metal porphyrin is used to functionalize the MoS₂ flakes 212, and the fine structure thereof can be seen in FIG. 2B.

In addition, a pair of electrodes formed at both ends of the gas sensing portion 200 is spaced apart from each other at a distance at which they commonly come in contact with one or more MoS₂ flakes 212. As described above, if a pair of the electrodes are spaced apart from each other at a distance corresponding to half or less of the average length of the MoS₂ flakes 212 included in the gas sensing portion 200, it is possible to eliminate an operation of precisely aligning the electrodes.

In an example of the present invention, cobalt tetraphenylporphyrin (Co-TPP) was used as metal porphyrin. Cobalt tetraphenylporphyrin (Co-TPP) can functionalize MoS₂ flakes 212 for benzene among many volatile organic compounds. For reference, benzene that is the most well-known volatile organic compound is not only a carcinogenic substance, but also a toxic substance that increases the possibility of development of various diseases, including aplastic anemia, acute leukemia, and bone marrow abnormalities.

In order to confirm the performance of the gas sensor 10 functionalized for benzene, the sensitivities to benzene of a gas sensor comprising a gas sensing portion formed of a Pristine MoS₂ cluster and the gas sensor 10 comprising the MoS₂ cluster 210 functionalized with cobalt tetraphenylporphyrin (Co-TPP) were comparatively tested.

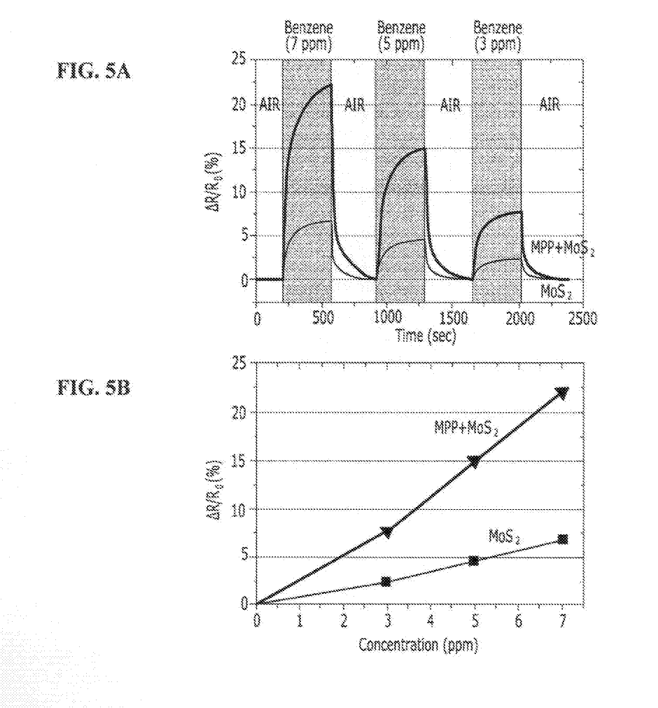

FIGS. 5A and 5B are graphs showing the results of the comparative tests. Specifically, FIG. 5A shows the results of measuring the sensitivity (ΔR/R₀) to 7, 5 and 3 ppm of benzene in real time. As can be seen in the graph, the sensitivity of the functionalized gas sensor 10 to benzene is significantly higher than that of the gas sensor comprising the pristine MoS₂ cluster. For example, it can be seen that the sensitivity of the functionalized gas sensor 10 to 3 ppm (the lowest concentration) of benzene is higher than the sensitivity of the gas sensor comprising the pristine MoS₂ cluster to 7 ppm (the highest concentration) of benzene.

In addition, the gas sensor 10 functionalized for benzene reaches a level corresponding to the highest sensitivity of the non-functionalized gas sensor within a short time. This indicates that the time required to detect benzene in response to benzene can be reduced. This greatly contributes to improvement in the performance of the gas sensor 10.

In addition, FIG. 5B shows the results of comparing the highest sensitivities of the gas sensors at each benzene concentration. It can be seen that the sensitivities to benzene of the functionalized gas sensor 10 at all the benzene concentrations are about two times higher than those of the gas sensor comprising the pristine MoS₂ cluster, and that the linearity of the sensitivities at a certain level or higher of the benzene concentration is also maintained.

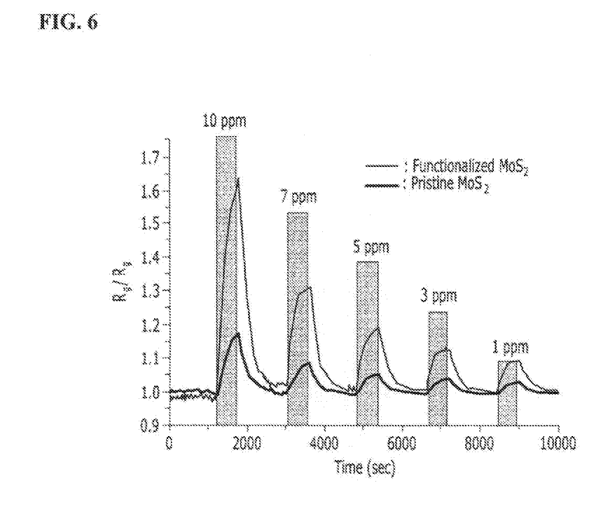

FIG. 6 is a graph showing the results of testing the sensitivity (ΔR/R₀) to toluene in real time in a manner similar to the test performed for benzene. The sensitivities (ΔR/R₀) to 10, 7, 5, 3 and 1 ppm of toluene were tested, and as a result, it was shown that the sensitivities of the functionalized gas sensor 10 to toluene was also significantly higher than those of the gas sensor comprising the pristine MoS₂ cluster. The sensing performance of the functionalized gas sensor 10 at all the toluene concentrations was about 30% higher than that of the gas sensor comprising the pristine MoS₂ cluster. When comparing with the results of the test performed for benzene, the sensitivity to toluene of the gas sensor 10 comprising the MoS₂ flakes or cluster functionalized with cobalt tetraphenylporphyrin (Co-TPP) was somewhat low, but it is evident that the ability of the gas sensor 10 to detect toluene was significantly improved by the functionalization. Particularly, the improved ability of the functionalized gas sensor 10 to detect both benzene and toluene that are representative VOCs can be considered a very positive result in terms of general use as a gas sensor.

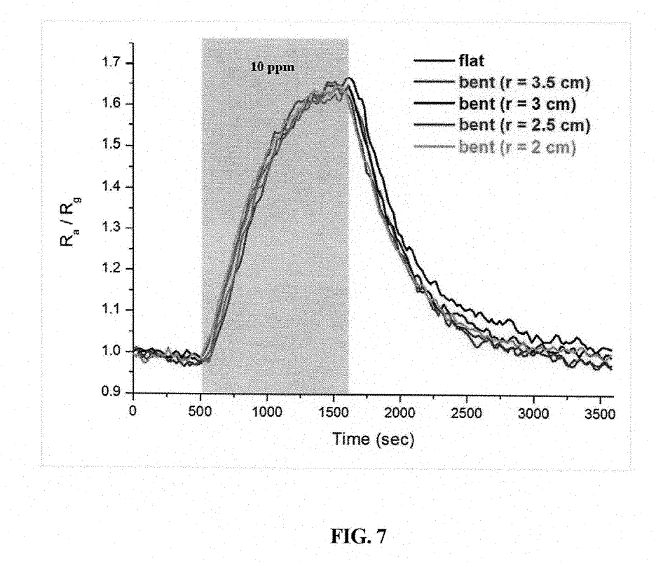

FIG. 7 shows the results of testing whether the sensitivity to toluene of the gas sensor 10 comprising the substrate 100 made of a transparent and flexible PET material would be somewhat changed or reduced when bending deformation was artificially applied to the gas sensor 10 (see FIG. 4). In FIG. 7, “flat” indicates no deformation, and the remainder indicates the results obtained when the amount of deformation was gradually increased such that the radius of curvature would decrease by 0.5 cm in the range of 3.5-2 cm. As shown in FIG. 7, the sensing performance of the gas sensor comprising the PET substrate 100 was not greatly reduced when the gas sensor 10 was significantly bent. This indicates that the gas sensor 10 of the present invention is very suitable to be mounted in various devices, particularly miniaturized wearable devices.

As described above, according to the present invention, it is possible to significantly increase the reaction sensitivity and reaction rate of the gas sensor for specific volatile organic compounds by functionalizing the MoS₂ flakes.

In the method for manufacturing the gas sensor according to the present invention, the gas sensor is manufactured by mixing solutions to functionalize the MoS₂ flakes, dropping droplets of the mixture solution onto the substrate, drying the dropped droplets, and then farming a pair of electrodes so as to be spaced apart from each other at a distance corresponding to half or less of the average length of the MoS₂ flakes. Thus, the method of the present invention is very suitable to produce a large amount of the gas sensor in a cost-effective manner.

Although the preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A gas sensor comprising:
an insulating substrate;
a gas sensing portion immobilized on the substrate and comprising MoS₂ flakes containing metal porphyrin; and
a pair of electrodes formed at both ends of the MoS₂ flakes of the gas sensing portion so as to be spaced apart from each other.

2. The gas sensor of claim 1, wherein the gas sensing portion is formed of a MoS₂ cluster consisting of a plurality of the MoS₂ flakes.

3. The gas sensor of claim 2, wherein the pair of electrodes are formed at both ends of the gas sensing portion so as to be spaced apart from each other at a distance at which they commonly come in contact with one or more of the MoS₂ flakes.

4. The gas sensor of claim 3, wherein the pair of electrodes is spaced apart from each other at a distance equal to half or less of the average length of the MoS₂ flakes.

5. The gas sensor of claim 1, wherein the substrate is made of a flexible and transparent material.

6. The gas sensor of claim 1, wherein the metal porphyrin is cobalt tetraphenylporphyrin (Co-TPP).

7. The gas sensor of claim 6, wherein the gas sensing portion has an increased sensitivity (ΔR/R₀) to benzene or toluene compared to a gas sensing portion formed of pristine MoS₂ flakes containing no cobalt tetraphenylporphyrin.

8. The gas sensor of claim 7, wherein the sensitivity (ΔR/R₀) of the gas sensing portion is at least 2 times higher to benzene and 30% higher to toluene.

9. A method for manufacturing a gas sensor, comprising the steps of:
(a) mixing a solution containing a plurality of MoS₂ flakes with a metal porphyrin-containing solution to prepare a mixture solution;
(b) Placing droplets of the mixture solution on an insulating substrate, and drying the placed droplets, thereby forming a gas sensing portion; and
(c) forming a pair of electrodes at both ends of the gas sensing so as to be spaced apart from each other at a distance at which they commonly come in contact with one or more of the MoS₂ flakes.

10. The method of claim 9, wherein the pair of electrodes is formed so as to be spaced apart from each other at a distance equal to half or less of the average length of the MoS₂ flakes.

11. The method of claim 10, wherein the pair of electrodes are formed at both ends of the gas sensing portion in any direction.

12. The method of claim 9, wherein the substrate is made of a flexible and transparent material.

13. The method of claim 9, wherein the metal porphyrin is cobalt tetraphenylporphyrin (Co-TPP), and the gas sensing portion has an increased sensitivity to benzene or toluene compared to a gas sensing portion formed of a pristine MoS₂ cluster containing no cobalt tetraphenylporphyrin.
